(19) United States
(12) Patent Application Publication
(10) Pub. No.: US 2011/0245503 A1
(43) Pub. Date: Oct. 6, 2011

(54) ENANTIOSELECTIVE SYNTHESIS OF ASYMMETRIC BETA-CARBOLINE INTERMEDIATES

(75) Inventors: Leonardo Santos, Talca (CL); Marlene Espinoza Moraga, Talca (CL); Nagula Shankarajah, Talca (CL)

(73) Assignee: UNIVERSIDAD DE TALCA, Talca (CL)

(21) Appl. No.: 13/131,751

(22) PCT Filed: Nov. 27, 2009
(86) PCT No.: PCT/CL2009/000025

§ 371 Date: May 27, 2011

(30) Foreign Application Priority Data
Nov. 28, 2008 (CL) 3573-2008

(51) Int. Cl. C07D 471/04; C07D 215/233

(52) U.S. Cl. 546/84; 204/157.71

(57) ABSTRACT

Described herein is a new asymmetric synthesis of imines to obtain β-carboline derivatives useful as key intermediate compounds for the synthesis of phosphodiesterase inhibitors using a new process with palladium or ruthenium hydride and/or nickel boride to reduce chiral intermediates. The use of chloroformate chiral auxiliaries is further described for the reduction and asymmetric hydrogenation of imines to obtain β-carboline derivatives and intermediate compounds used in the preparation thereof.

The present invention relates to an improved synthesis of a series of asymmetric β-carboline intermediates for obtaining pyrrole quinolones that can inhibit phosphodiesterase type 5 and increase cGMP concentration in fine cavernous tissue in vitro.

### PRIOR ART

Substituted pyrrolopyridinone derivatives are known as phosphodiesterase (PDE) inhibitors useful for the treatment of diseases and conditions related thereto. The diseases associated with PDE inhibitors include male erectile dysfunction, female sexual dysfunction, cardiovascular disease, atherosclerosis, blood disorders, thrombosis, coronary stenosis, angina pectoris, myocardial infarction, heart failure, ischemic heart disorder, hypertension, pulmonary hypertension, asthma, and diabetic complications, among others. Particularly, male erectile dysfunction (ED) occurs when there is in ability to achieve or maintain an erection firm enough for sexual intercourse. Approximately 10% of the world's male population has been estimated to suffer from some degree of ED. The incidence of this disorder increases with the age of the individual. As average age and life expectancy increase, a larger number of people will be affected by this disorder in the future.

One of the PDE inhibiting compounds widely used mainly for the treatment of ED is sildenafil (Viagra®, [5-(2-ethoxy-5-(4-methylpiperazine-1-ylsulfonyl)phenyl)-1-methyl-3-N-propyl-6,7-dihydro-1H-pyrazolo(4,3-d)pyrimidin-7-one]) and analogs thereof, which have been described in U.S. Pat. No. 5,250,534 and U.S. Pat. No. 5,346,901, and the use thereof for treating ED has been described in WO 94/28902. Studies have shown that sildenafil and the analogs thereof inhibit the PDE type 5 enzyme (PDE5) which increases nitric oxide induced cGMP (Brook, 2000). These compounds were initially researched for the treatment of angina (Corbin et al., 1999), and later proved to be effective in treating ED. However, despite their effectiveness, some clinical data suggest that the intake of these compounds causes side effects such as headache, nausea, flushing and visual disturbances (Gresser et al., 2002). In addition, sildenafil and other PDE5 inhibitors are contraindicated for patients taking nitrates or NO donors because there is a decrease in blood pressure after co-administration of both compounds (Kloner, 2000). At the same time, other molecules have been reported to have the ability to inhibit PDE5 and are used for different applications and conditions. Such is the case of tetracyclic pyrroloquinolone derivatives (WO 95/19978, EP 1448562, EP 0740668, U.S. Pat. No. 6,784,179, U.S. Pat. No. 6,369,059, U.S. Pat. No. 6,143,746, U.S. Pat. No. 6,127,542, U.S. Pat. No. 6,025,494 and U.S. Pat. No. 5,859,009), 9H-pyrrolo[3,4-b]quinolin-9-ones (DE 2803541, U.S. Pat. No. 4,235,907) and the synthesis of tricyclic quinolone derivatives (Garinaux et al., 1997).

Several ways to synthesize PDE inhibitors have been reported, such as catalytic reduction of imines and carbonyl in heterocyclic compounds (Ryoji Noyori, a pioneer in asymmetric synthesis, wherein the result of the reduction could be predicted depending on the desired isomers).

Additionally, Willemsens et al. (2006) discloses the process for preparing (3R)-3-(2,3-dihydro-1-benzofuran-5-yl)-1,2,3,4-tetrahydro-9H-pyrrolo[3,4-b]quinolin-9-one, wherein a 1-(2,3-dihydro-benzofuran-5-yl)-2,3,4,9-tetrahydro-1H-β-carboline of formula (6) is reacted with benzyl chloroformate, triethylamine and ethyl acetate to yield 3-(2,3-dihydro-benzofuran-5-yl)-1,2,3,4-tetrahydro-pyrrolo[3,4-b]quinolin-9-ona (10) as shown below.

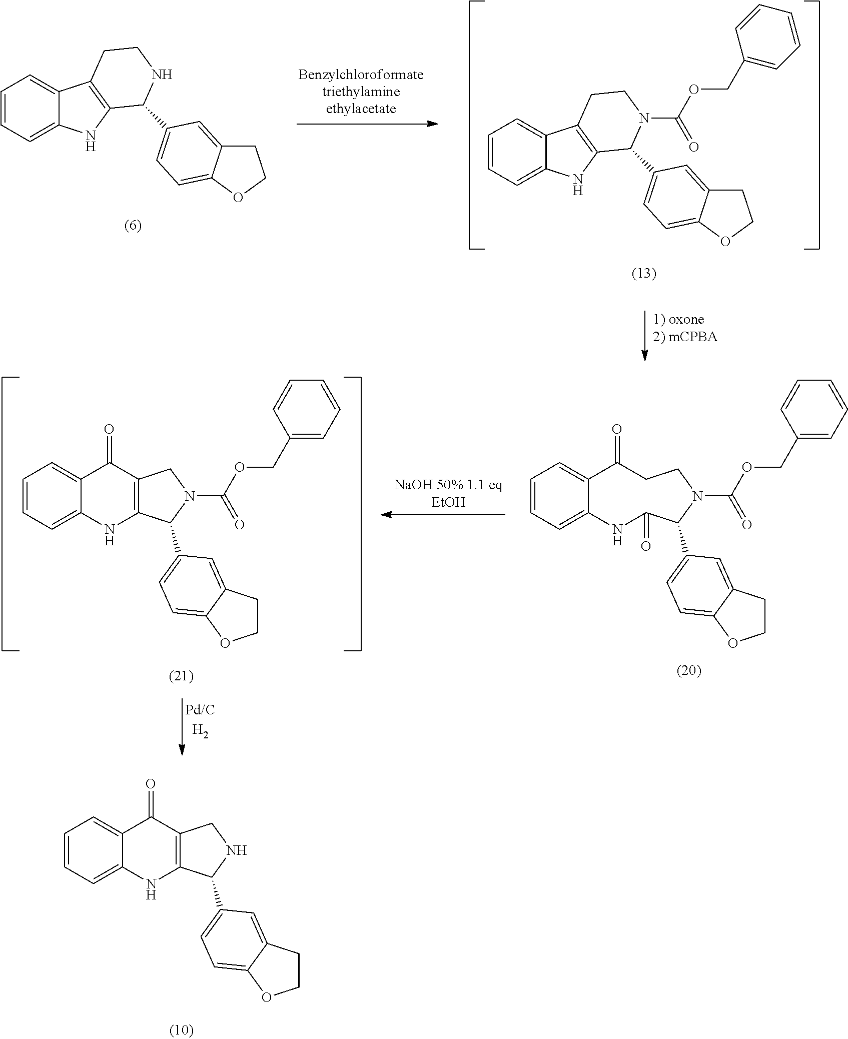

U.S. 2007/0015798 discloses a process for large-scale synthesis of (3R)-3-(2,3-dihidrobenzofuran-5-yl)-1,2,3,4-tetrahydro-pyrrolo[3,4-b]quinolin-9-one derivatives and key intermediates used for the preparation of benzofuranyl pyrroloquinolones, and describes the reaction of 1-(2,3-dihydrobenzofuran-5-yl)-2,3,4,9-tetrahydro-1H-β-carboline with MeOH/EtOAc to give intermediate compound (4) as shown below, which is reacted with bromomethyl benzene in K₂CO₃ and CH₂Cl₃ to give 2-benzyl-1-(2,3-dihydro-benzofuran-5-yl)-2,3,4,9-tetrahydro-1H-β-carboline (7). However, the aforementioned process is not stereoselective, as it starts from the resolution of the racemic mixture of compound (4).

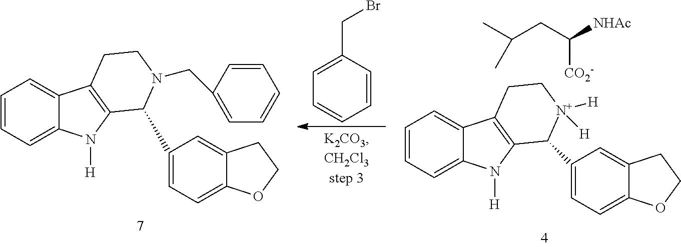

Similarly, WO 2004/000842 and EP 1534707 describe the process for the preparation of 2-benzyl-3-(2,3-dihydrobenzofuran-5-yl)-2,4-dihydropyrrolo[3,4-b]quinolin-9-one, wherein the starting material is 2,4-dihydropyrrolo[3,4-b]quinolin-9-one substituted at position 2 by an aryl group, as shown below.

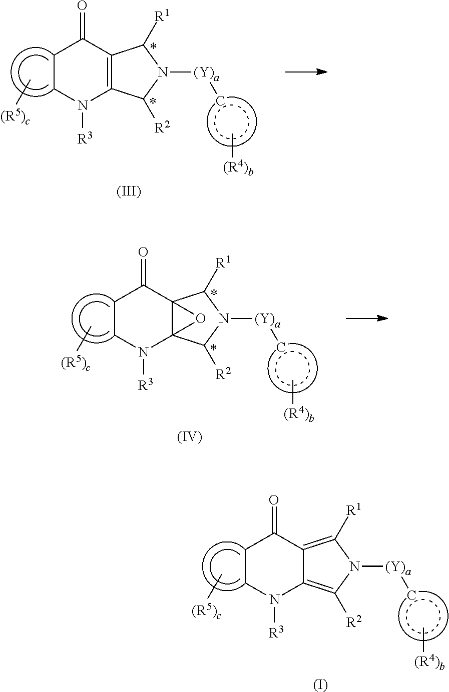

Finally, Shankaraiah et al. (2008) and Santos (2003) describe asymmetric syntheses for incorporating chirality in molecules. In addition, Jiang et al. (2004) describes the synthesis of tetracyclic fused pyrroloquinolones in four steps. Jiang et al. (2003) describes an alternative to Winterfeldt's reaction by using potassium superoxide (KO₂) according to the following reaction scheme:

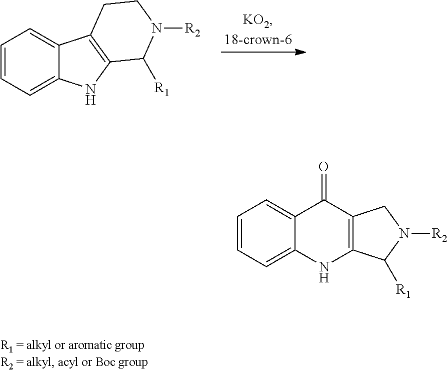

Li et al. (2005), Lemaire et al. (2007), Tsuji et al. (2003) and Jiang et al. (2002) performed the diastereoselective synthesis of molecules using Pictet-Spenger's and/or Winterfeldt's reaction. Thus, although prior art references disclose several enantioselective synthetic processes for 3-(2,3-dihydro-benzofuran-5-yl)-1,2,3,4-tetrahydro-pyrrolo[3,4-b]quinolin-9-one, none of them is equivalent to the process of the present invention.

Previous methodologies for the synthesis of pyrrole quinolones used the resolution of the racemic mixture of β-carbolines by chiral separation of salts and complexes. Under these conditions, the yield of the process may be only 50% of the final product. Despite the importance of chirality in several fields such as medicine, there are only a few methodologies that adopt a stereoselective approach to obtain the final products.

### DESCRIPTION OF THE INVENTION

The present invention relates to a new asymmetric synthesis of imines to produce β-carboline derivatives useful as key intermediate compounds for the synthesis of phosphodiesterase inhibitors using a new synthesis process with palladium or ruthenium hydride and/or nickel boride to reduce chiral intermediates. The invention further relates to the use of chloroformate chiral auxiliaries for the reduction and asymmetric hydrogenation of imines to produce β-carboline derivatives and intermediate compounds used in the preparation thereof.

The present invention describes an efficient synthetic route comprising 5-6 steps for obtaining functional intermediate compounds as PDE5 inhibitors of the pyrroloquinolone type. The present invention can be used on an industrial scale and is scalable because of its innovative qualities and the operational simplicity of the process. The most striking features of the present invention include high diastereoselectivity and enantioselectivity based on a hydrogenation reaction with chiral auxiliaries.

Furthermore, the invention provides an enantiomeric synthesis of compounds capable of generating an increased concentration of cGMP in penile tissue and corpus cavernosum by inhibiting PDE, specifically PDE5.

### PREFERRED EMBODIMENTS OF THE INVENTION

The present invention is concerned with the synthesis of β-carbolines, wherein an alternative synthesis route has been developed for PDE inhibitors, preferably PDE5, by obtaining key intermediate compounds for the synthesis of said inhibitors. Key intermediates correspond to the compounds of formula (II). For example, in a preferred embodiment, said compounds correspond to the intermediate of formula (2). Moreover, PDE inhibitors comprise a tricyclic structure which is characteristic of pyrroloquinoline as described for the compound of formula (I), wherein, for example, a preferred embodiment corresponds to the compound of formula (1), wherein the intermediate compounds of the invention are relevant for the preparation thereof.

Therefore, the present invention relates to PDE inhibitors of formula (I)

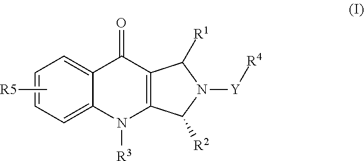

wherein:

R¹ is selected from the group consisting of H; ═O, carboxyl, cyano, amino, halogen; lower alkyl or alkene or alkenyl or alkoxy or alkylamine comprising 1-6 carbon atoms, unsubstituted or substituted preferably with halogen, cyano, amino, and hydroxy, among others; —C(O) (C₁₋₆) alkyl, —C(O)— (C₁₋₆)alkoxy, —C(O)—NH—(C₁₋₆)alkylamino or —C(O)—NH₂.

R² is selected from the group consisting of H, ═O, carboxyl, cyano, amino, halogen, trifluoromethyl, trifluoromethoxy, (C₁₋₁₀)alkyl or alkene or alkenyl or alkoxy or alkyl, unsubstituted or substituted preferably with halogen, cyano, amino, hydroxy, trifluoromethyl, and trifluoromethoxy, among others; bipyridine, and pyridine substituted with alkyl, alkene, halide, and carboxy; benzyl substituted with alkyl, alkene, halide, carboxy, carbamate, nitro, alkyl, alkene, and carboxy; cycloalkyl or heteroaryl or heterocycloalkyl unsubstituted or substituted with O, S, P, halogen, cyano, amino, hydroxy, lower aryl or alkyl or alkene or alkenyl or alkoxy or alkylamine, trifluoromethyl, and trifluoromethoxy, among others.

R³ may be selected from CH₃; H; lower alkyl or alkene or alkynyl or carbonyl alkyl, carbonyl alkynyl or carbonyl alkene; sulfates; phenyl substituted with alkyl, alkene, halide, and carboxy; pyridine, and bipyridine substituted with alkyl, alkene, halide, and carboxy; benzyl substituted with alkyl, alkene, halide, carboxy, carbamate, nitro, alkyl, alkene, and carboxy, among others.

Y may be selected from a bond, lower alkyl or alkenyl or alkynyl, or a heteroatom, phosphates or phosphites.

R⁴ can be selected from the group consisting of pyridine, and bipyridine substituted with alkyl, alkene, halide, carboxy, chlorobenzyl substituted with alkyl, alkene, halide, carboxy, carbamate, and nitro; chloroalkyl; benzyl substituted with alkyl, alkene, halide, carboxyl, carbamate, and nitro; alkyl; alkene; carboxy, chloroalkyl, and chloroalkene; acid chlorides; H, halogen, hydroxy, carboxy, oxo, nitro, lower alkyl or alkene or alkynyl or carbonyl alkyl or carbonyl alkene or carbonyl alkenyl or arylalkyl, heteroaryl, and heterocycloalkyl, whether substituted or not; Boc, Bn, phenyl or benzyl, whether substituted or not, phenylsulfonyl, and naphthyl.

R⁵ may be from 1 to 4 identical or different radicals selected from H, ═O, carboxy, cyano, amino, halogen, trifluoromethyl, trifluoromethoxy, (C₁₋₁₀) alkyl or alkene or alkenyl or alkoxy or alkylamine, unsubstituted or substituted preferably with halogen, cyano, amino, hydroxy, trifluoromethyl, and trifluoromethoxy.

In addition, R¹, R², R³, R⁴, and R⁵ may be selected from:

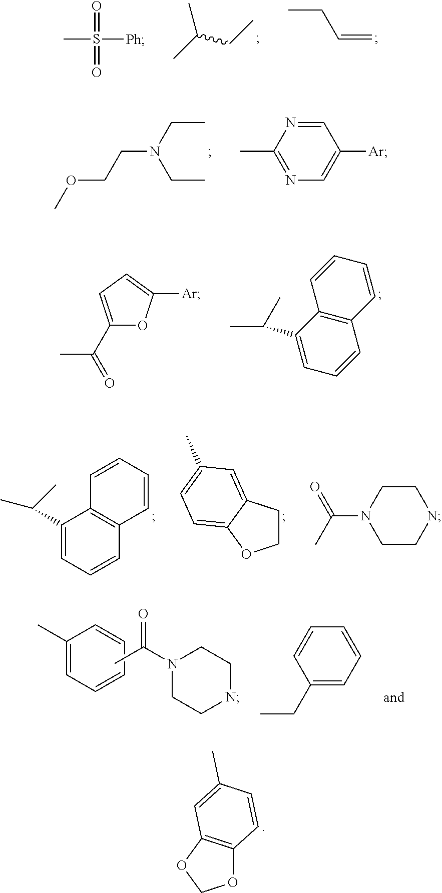

Furthermore, the present invention relates to intermediate compounds of formula (II)

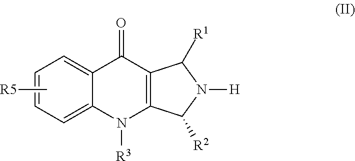

wherein:

R¹ is selected from the group consisting of H; ═O, carboxyl, cyano, amino, and halogen; lower alkyl or alkene or alkenyl or alkoxy or alkylamine comprising 1-6 carbon atoms, unsubstituted or substituted preferably with halogen, cyano, amino, and hydroxy, among others, —C(O)—(C₁₋₆)alkyl, —C(O)—(C₁₋₆)alkoxy, —C(O)—NH—(C₁₋₆)alkylamino or —C(O)—NH₂.

R² is selected from the group consisting of H, ═O, carboxyl, cyano, amino, halogen, trifluoromethyl, trifluoromethoxy, (C₁₋₁₀) alkyl or alkene or alkenyl or alkoxy or alkyl, unsubstituted or substituted preferably with halogen, cyano, amino, hydroxy, trifluoromethyl, and trifluoromethoxy, among others; bipyridine, and pyridine substituted with alkyl, alkene, halide, and carboxy; benzyl substituted with alkyl, alkene, halide, carboxy, carbamate, nitro, alkyl, alkene, and carboxy; cycloalkyl or heteroaryl or heterocycloalkyl unsubstituted or substituted with O, S, P, halogen, cyano, amino, hydroxy, lower aryl, alkyl or alkene or alkenyl or alkoxy or lower alkylamine, trifluoromethyl, and trifluoromethoxy, among others.

R³ may be selected from CH₃; H; lower alkyl or alkene or alkynyl or carbonyl alkyl, carbonyl alkynyl or carbonyl alkene; sulfates; phenyl substituted with alkyl, alkene, halide, and carboxy; pyridine and bipyridine substituted with alkyl, alkene, halide, and carboxy; benzyl substituted with alkyl, alkene, halide, carboxy, carbamate, nitro, alkyl, alkene, and carboxy, among others.

R⁵ may be from 1 to 4 identical or different radicals selected from H, ═O, carboxy, cyano, amino, halogen, trifluoromethyl, trifluoromethoxy, (C₁₋₁₀) alkyl or alkene or alkenyl or alkoxy or alkylamine, unsubstituted or substituted preferably with halogen, cyano, amino, hydroxy, trifluoromethyl, and trifluoromethoxy.

In addition to the foregoing descriptions, R¹, R², R³, R⁴, and R⁵ may be selected from:

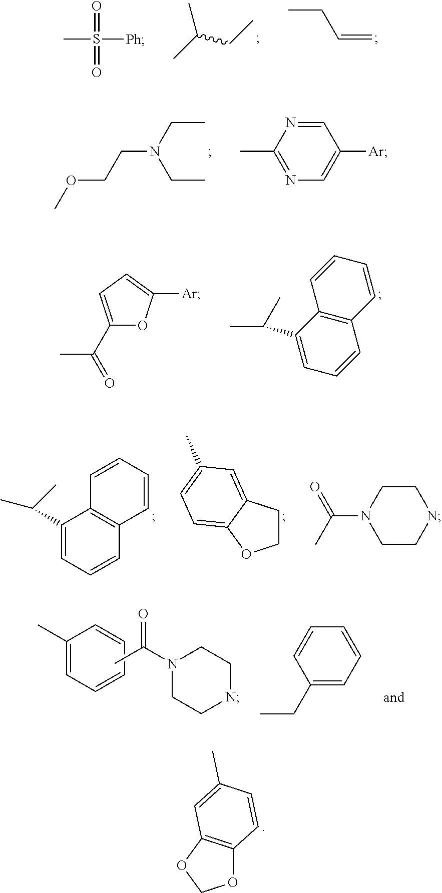

In a preferred embodiment of the invention, the compounds of formula (I) and the intermediate compounds of formula (II) are selected as compounds of formula (1) and (2) respectively.

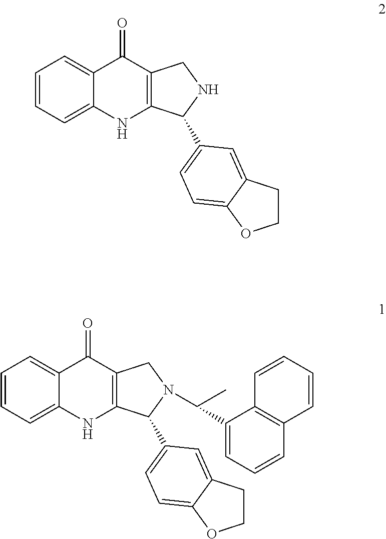

A further embodiment of the present invention relates to the method of synthesizing both PDE inhibitors, preferably PDE5, and intermediate compounds as described above which fit the description of the compounds of formula (I) and (II) respectively. The synthesis method provides high diastereoselectivity and enantioselectivity, which translates into improved efficiency in terms of the specific production of preferred enantiomers. The synthesis process in accordance with the present invention increases the amount of intermediate compounds described above, thus offering an efficiency greater than 60%, preferably about 75%, more preferably about 90%, and most preferably about 99% of the preferred enantiomer, which increases the total yield of the final product by 200% as compared to the methods described in the prior art. This means that the present invention does not require the separation or resolution of racemic intermediates or final products to improve the efficiency of the synthesis.

Said efficiency is achieved because the present invention discloses the use of chloroformate chiral auxiliaries preferably selected from 8-phenyl-mentholyl chloroformate, mentholyl chloroformate, abietane chloroformate, ciperenoic acid chloroformate, trans-phenylcyclohexanol chloroformate, and ferruginol chloroformate to obtain the desired pyrroloquinolone as described in Figure 1.

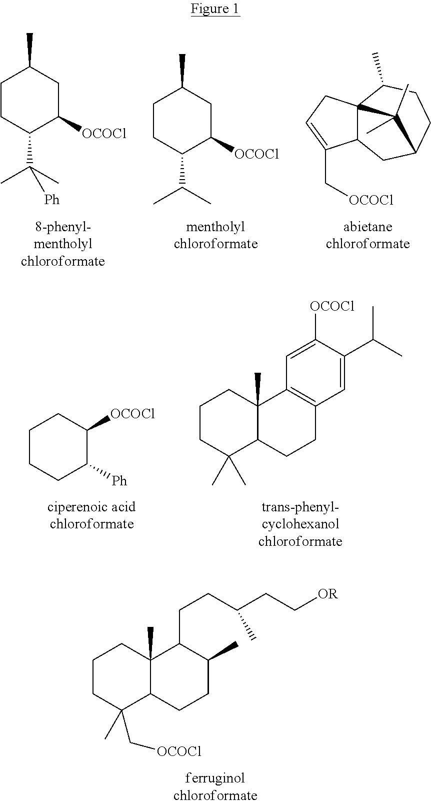

These chiral auxiliaries are used for obtaining PDE inhibiting compounds of formula (I), and specially for obtaining the intermediate compounds of formula (II) according to the present invention. These intermediates of formula (II) are key to the diastereoselective and enantioselective synthesis of the PDE inhibitors according to the present invention. This process is depicted in a preferred embodiment in Figure 2, wherein the compounds of formula (6), which are precursors of the compounds of formula (I) and (II), are reacted with chloroformate as described above. For illustrative purposes and for the sake of simplicity, R¹, R³ and R⁵ are all H, while R² is selected as shown in the figure and, in a preferred embodiment, R* is selected from the indicated chloroformates. The precursors of formula (6) are reacted with the chloroformate compounds of formula (11) to yield the compounds of formula (12) and finally the intermediate compounds of formula (II), such as the preferred compound of formula (2) as shown in Figure 2.

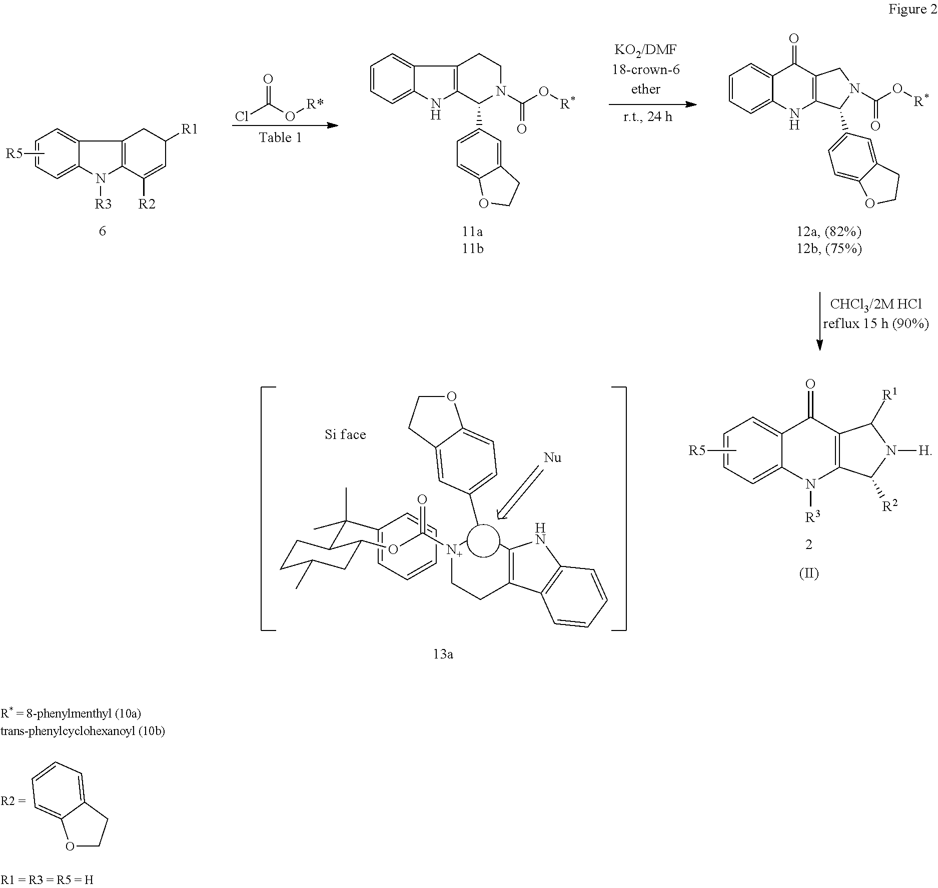

Accordingly, the invention also comprises a method for obtaining PDE inhibitors and intermediate compounds corresponding to the compounds of formula (I) and/or (II), respectively, through a process that does not require racemic separation of final products for significantly enriching the desired enantiomer. The intermediate compounds of formula (II) can be converted into PDE inhibitors of formula (I) under conditions generally known to a person having ordinary skill in the art and described in the prior art as referenced in the instant application, for example in WO 2004/000842 and U.S. 2007/0015798, which are cited herein by way of reference. However, the present invention also comprises a modification for obtaining inhibitory compounds of formula (I) from intermediate compounds of formula (II).

In a preferred embodiment, in the final step of the process, the introduction of radicals is performed according to the

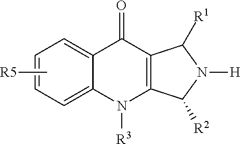

scheme shown in Figure 3, by microwave reaction as shown in step 1 in Figure 3. Chlorides are preferably used as precursors. The process involves the use of PdCl₂ catalyzed Buchwald-Hartwig's reaction catalyzed and ionic liquids such as [bmim] Z (wherein Z is selected from PF₆, CF₃, CO₂, BF₄, InCl₄, and BPh₄). The addition of YR⁴ in (II) can be performed with chloropyridine and chlorobipyridine substituted with alkyl, alkene, halide, carboxy, chlorobenzyl substituted with alkyl, alkene, halide, carboxy, carbamate, and nitro; chloroalkyl; benzyl substituted with alkyl, alkene, halide, carboxy, carbamate, and nitro; alkyl; alkene; carboxy; chloroalkyl; chloroalkene; acid chloride; H, halogen, hydroxy, carboxy, oxo, nitro, substituted or unsubstituted lower alkyl or alkene or alkynyl or carbonyl alkenyl or carbonyl alkene or carbonyl alkynyl or arylalkyl, heteroaryl, heterocycloalkyl; Boc, Bn, phenyl or benzyl, whether substituted or not, phenylsulfonyl, and naphthyl.

### Figure 3

According to the present invention, the compound of formula (6) and the derivatives thereof according to Figure 2 are obtained

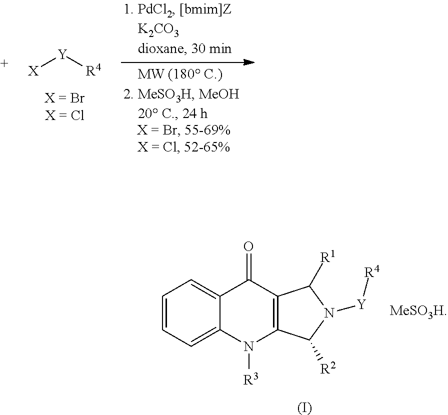

according to conditions and steps generally known to one skilled in the art, as referred in this application, and incorporated herein by reference.

Upon obtaining the compound of formula (6) and the derivatives thereof, the synthesis is modified as previously described, using chloroformate chiral auxiliaries. The compound of formula (6) is converted into the compound of formula (11) at room temperature, preferably between 15 to 40° C., for 15 to 300 minutes, preferably between 30 and 60 minutes. Afterwards the temperature is reduced to below 0° C., preferably below −20° C., more preferably less than −50° C., and preferably about −80° C. Later a reduction with Pd—H is carried out at a temperature between 0° C. and room temperature for 1 to 24 hours, preferably between 1 and 12 hours, preferably about 2-3 hours. Preferably, reducing reagents are used, which are selected, for instance, from PdCl₂/Et₃SiH, NaCNBH₃, RuCl₂/HCO₂H:Et₃N in the presence of CH₂Cl₂/THF, CH₂Cl₂, and MeCN as solvents.

Having obtained the compound of formula (11), it is subsequently converted into the compound of formula (12). The re-action is performed at room temperature as described above, wherein crown ethers are used in an oxidizing environment, preferably a 18-crown-6 ether or an equivalent thereof. The reaction is run from 1 to 30 hours, preferably between 2 and 20 hours, using preferably between 3 and 16 equivalents of the oxidizing agent, preferably Na₂O₂, for each equivalent of the crown ether.

Finally, the compound of formula (12) is converted into the compound of formula (II), preferably the compound of formula (2), using acid treatment at elevated temperature, preferably under reflux conditions, for a period exceeding 10 hours, preferably between 12 and 20 hours, and more preferably about 15 hours.

The preceding process corresponds to an explanation of the general process using specific compounds, and is neither intended to restrict nor limit the scope of the invention.

### Example 1

### Chiral Auxiliary-Mediated Reduction

To obtain the intermediate compounds of formula (II), preferably the compound of formula (2), a retro-synthesis analysis was performed based on the structure of the compound of formula (1) and the characteristics of the chiral auxiliary-mediated reduction as a key step (Figure 4).

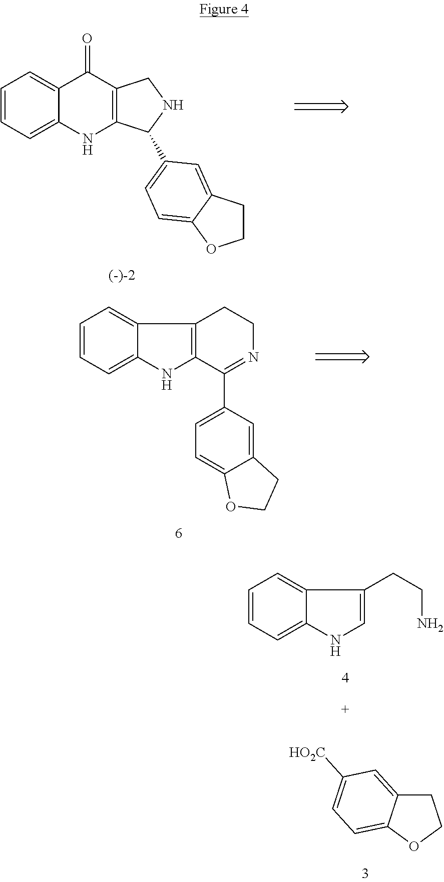

In order to obtain the imine of formula (6), dihydrobenzo[b]furan-5-carboxylic acid of formula (3) and tryptamine were used together with EDC/HOBt, which yielded the corresponding amide of formula (5), followed by Bischler-Napieralsky cyclization according to Figure 5.

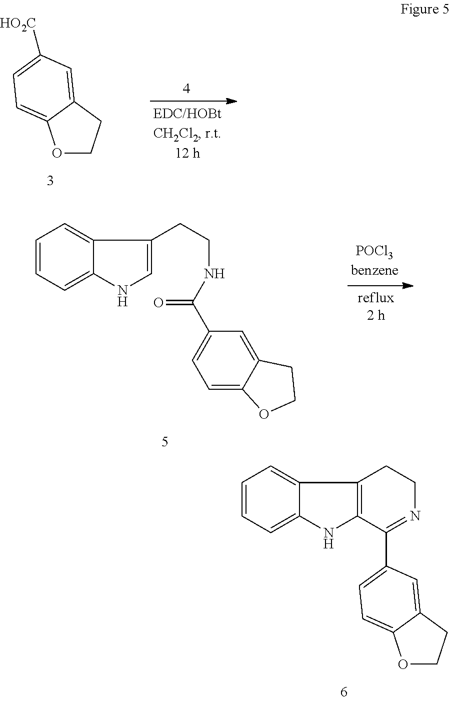

For the purposes of ensuring an adequate stereogenic center in the dihydro β-carboline, the α-phenylmethyl chloroformate of formula (10a) and the trans-phenylcyclohexyl of formula (10b) were used as shown in Figure 2. According to the above, the in situ formation of the corresponding N-acyliminium ion (11a,b) was achieved by adding chiral auxiliary chloroformate to the compound of formula (6) at room temperature. The temperature was decreased to −78° C. and then a reduction with Pd—H was performed following the PdCl₂/Et₃SiH protocol according to Sakaitani et al., 1990. As shown in Table 1, the yield using (11a) is 85%, while the yield using (11b) is 78% (lines 1 and 2 respectively).

The same protocol was followed with NaCNBH₃ (90 and 87% yield respectively for (11a) and (11b), lines 3 and 4, Table 1) in CH₂Cl₂/THF (2:1) as solvent at room temperature. Additionally, upon using NaBH₄ in CH₂Cl₂/THF (2:1) and performing the reaction between 0° C. and room temperature for 2 hours, a yield of 90 and 82% was obtained for (11a) and (11b) respectively (lines 5 and 6 in Table 1). Finally, when using RuCl₂ as a reducing agent and HCO₂H:Et₃N, a yield of 85-97% was obtained depending on the auxiliary agent being used (lines 7 and 8). All optical rotations were measured with Pd—H chiral auxiliaries, NaCNBH₃, NaBH₄, and Ni₂B respectively, and correspond to the following values obtained with the chiral auxiliary: 11a [α]_{D}−14.0 (c=1.0, MeOH), 11b [α]_{D}−44.1 (c=MeOH) with PdH, 11a [α]_{D}−34.25 (c=1.0, MeOH), 11b [α]_{D}−19.5 (c=1.0, MeOH) with NaCNBH₃ and 11a [α]_{D}−24.5 (c=1.0, MeOH), 11b [α]_{D}−50.6 (c=1.0, MeOH) with NaBH₄, respectively.

The configuration of these compounds was determined to be (R) after the chiral auxiliary was removed using HCl/CHCl₃ (6 M), thus giving (+)−7 with a yield of 89%, [α]_{D}+46.9 (c=1.0, MeOH); the enantiomeric excess for (+)−7 was 99% as determined by HPLC, which is consistent with (R)-(+)−7. The enantiomeric excess analysis was performed by HPLC using a ChiralPack column. After deprotection of (R)-11b obtained by reduction of (6) using (10b) and NaBH₄, (+)−7 with [α]_{D})+26 (c=0.04, MeOH) was observed.

According to the results of this example as shown in Table 1, the most efficient solvent ratio to obtain the desired products is CH₂Cl₂/THF (2:1). In addition, the method of the invention provides yields above 90% when the reaction is carried out with NaBH₄ at room temperature and decreases the ee % to 52% and 44% in the presence of (11a) or (11b) respectively. Moreover, the chiral auxiliaries were recovered at a rate above 90%, preferably greater than 95%, without reducing optical rotation. At the same time, the selectivity obtained with the chiral auxiliary in the reduction of the compound of formula (6) was rationalized by the transition state shown in Figure 6, which is consistent with the reduction of the N-acyliminium ion (13) through the Si-face.

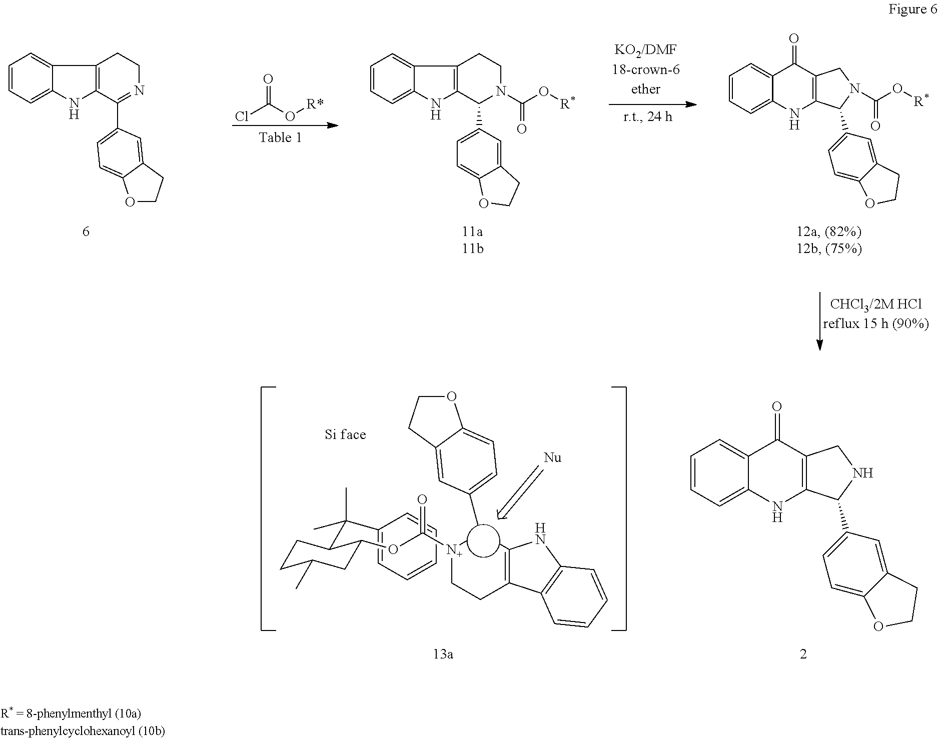

TABLE 1

| | Chiral | | | |
|---|---|---|---|---|
| Case | auxiliary | Reaction conditions ^{c} | Yield (%)^{a} | dr (%)^{b} |
| 1 | 10a | A | 85 | 14:1 |
| 2 | 10b | A | 78 | 7:1 |
| 3 | 10a | B | 90 | 10:1 |
| 4 | 10b | B | 87 | 7:1 |
| 5 | 10a | C | 90 | 6:4 |
| 6 | 10b | C | 82 | 4:1 |
| 7 | 10a | D | 85 | 11:1 |
| 8 | 10b | D | 97 | 8:1 |

(a) Yield.
(b) Enantiomeric excess (ee %) as calculated by HPLC.
(c) A: chiral auxiliary, CH₂Cl₂, rt, 30 min., then PdCl₂/Et₃SiH, −78° C., 1 h;
B: chiral auxiliary, CH₂Cl₂, rt, 30 min., then NaCNBH₃/THF, −78° C., 1 hr.;
C: chiral auxiliary, CH₂Cl₂, rt, 30 min., then NaBH/THF, 0° C., rt, 1 h;
D: chiral auxiliary, CH₂Cl₂, rt, 30 min., then RuCl₂ and HCO₂H:Et₃N (5:2, v/v), 0° C., 12 h.

### Example 2

### Chiral Auxiliary-Mediated Reduction

In one embodiment of the present invention, the intermediate compound for the synthesis of S-(−)-quinolactacin B was also obtained using the methodology described in Example 1. In this case, a high yield was obtained with enantiomeric excesses of about >94% ee.

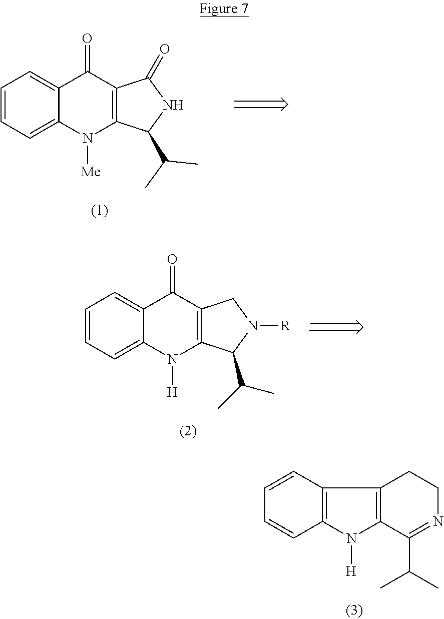

### Comparative Example

### Noyori's Asymmetric Hydrogenation Reaction

The imine of formula (6) was obtained with a yield of 75% from the dihydrobenzo[b]furan-5-carboxylic acid of formula (3) and tryptamine using EDC/HOBtl, which gave the corresponding amine of formula (5), followed by Napieralsky Bischler's cyclization. Having prepared the imine of formula (6), the next step was to achieve the required asymmetry through Noyori's asymmetric hydrogenation, using p-cymene-Ru (II) complexes of certain chiral 1,2-diamines plus an azeotropic mixture of formic acid-triethylamine as a reducing agent. The Noyori's reduction of the imine of formula (6) is performed using a preformed complex of (S,S)-TsDPEN-Ru (II) in DMF and a mixture of HCO₂H-Et₃N, which gave the amine of formula (7) with a yield of 92%, as analyzed by HPLC with a ChiralPack OD column. In this case, the (R)-(+)−7 compound was obtained through the use of (S,S)-TsDPEN-Ru (II). This compound was subsequently protected with Boc anhydride and Et₃N in CH₂Cl₂, thus obtaining the compound (+)−8 with a yield of 98%. Then (+)−8 was subjected to Winterfeldt's reaction using KO₂ and 18-crown-6 ether, thus giving (+)−9 with a yield close to 85% after 16 hours. Finally, the compound of formula (2) was obtained at a yield of 94% by treatment with ZnBr₂. The process described above is depicted in Figure 8.

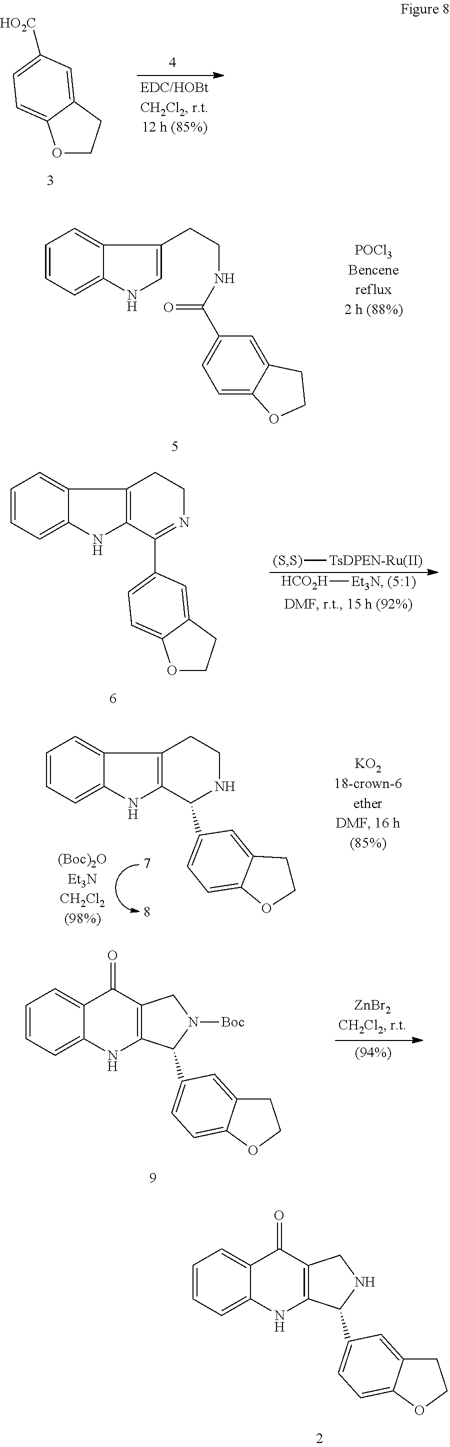

1. Method for the asymmetric synthesis of imines to obtain β-carbolines derivatives of formula (I) 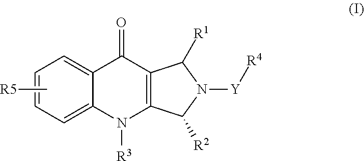
wherein:
R¹ is selected from the group consisting of H, ═O, carboxyl, cyano, amino, and halogen; lower alkyl or alkene or alkenyl or alkoxy or alkylamine comprising 1-6 carbon atoms, unsubstituted or substituted preferably with halogen, cyano, amino, and hydroxy, among others; —C(O)—(C₁₋₆)alkyl, —C(O)—(C₁₋₆)alkoxy, —C(O)—NH—(C₁₋₆)alkylamino or —C(O)—NH₂;
R² is selected from the group consisting of H, ═O, carboxyl, cyano, amino, halogen, trifluoromethyl, trifluoromethoxy, (C₁₋₁₀)alkyl or alkene or alkenyl or alkoxy or alkyl, unsubstituted or substituted preferably with halogen, cyano, amino, hydroxy, trifluoromethyl, and trifluoromethoxy, among others; bipyridine and pyridine substituted with alkyl, alkene, halide, and carboxy; benzyl substituted with alkyl, alkene, halide, carboxy, carbamate, nitro, alkyl, alkene, and carboxy; cycloalkyl or heteroaryl or heterocycloalkyl unsubstituted or substituted with O, S, P, halogen, cyano, amino, hydroxy, lower aryl, alkyl or alkene or alkenyl or alkoxy or lower alkylamine, trifluoromethyl, and trifluoromethoxy, among others;
R³ is selected from CH₃; H; lower alkyl or alkene or alkynyl or carbonyl alkyl, carbonyl alkynyl or carbonyl alkene; sulfates; phenyl substituted with alkyl, alkene, halide, and carboxy; pyridine and bipyridine substituted with alkyl, alkene, halide, and carboxy; benzyl substituted with alkyl, alkene, halide, carboxy, carbamate, nitro, alkyl, alkene, and carboxy among others;
Y may be selected from a bond, lower alkyl or alkenyl or alkynyl, or a heteroatom, phosphates or phosphites;
R⁴ is selected from the group consisting of pyridine and bipyridine substituted with alkyl, alkene, halide, carboxy, chlorobenzyl substituted with alkyl, alkene, halide, carboxy, carbamate, and nitro; chloroalkyl; benzyl substituted with alkyl, alkene, halide, carboxyl, carbamate and nitro; alkyl; alkene; carboxy, chloroalkyl, and chloroalkene; acid chlorides; H, halogen, hydroxy, carboxy, oxo, nitro, lower alkyl or alkene or alkynyl or carbonyl alkyl or carbonyl alkene or carbonyl alkenyl or arylalkyl, heteroaryl, and heterocycloalkyl, whether substituted or not; Boc, Bn, phenyl or benzyl, whether substituted or not, phenylsulfonyl, and naphthyl;
R⁵ may be from 1 to 4 identical or different radicals selected from H, ═O, carboxy, cyano, amino, halogen, trifluoromethyl, trifluoromethoxy, (C₁₋₁₀) alkyl or alkene or alkenyl or alkoxy or alkylamine, unsubstituted or substituted preferably with halogen, cyano, amino, hydroxy, trifluoromethyl, and trifluoromethoxy;
wherein, in addition, R¹, R², R³, R⁴, and R⁵ are selected from: 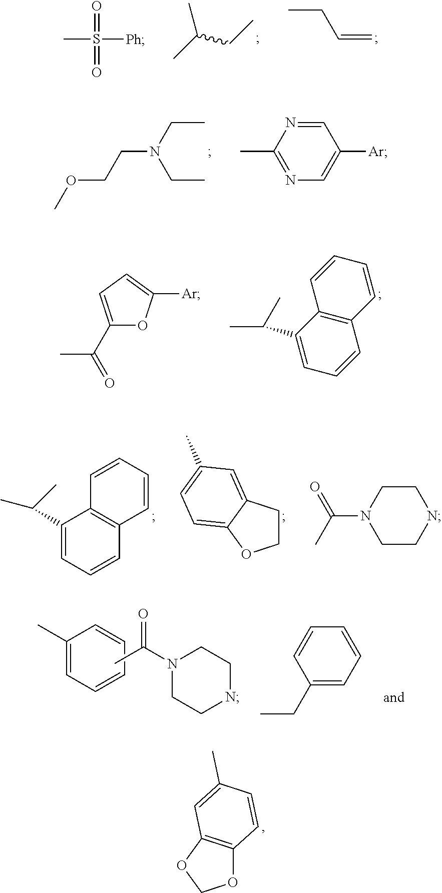
characterized in that it is an enantioselective and diastereoselective process for obtaining an enantiomerically enriched product, wherein a hydrogenation reaction is carried out with chiral auxiliaries, thus obtaining the desired enantiomeric product without requiring resolution of the racemic mixture of β-carbolines by chiral separation of salts and complexes.

2. Method according to claim 1, characterized in that palladium or ruthenium hydride, and/or nickel boride are used in the synthesis to reduce chiral intermediates.

3. Method according to any one of the preceding claims, characterized in that chloroformate chiral auxiliaries are used for the reduction and asymmetric hydrogenation of imines.

4. Method according to any one of the preceding claims, characterized in that the chloroformate chiral auxiliaries is selected from 8-phenyl-menthol, mentholyl chloroformate, abietane chloroformate, ciperenoic acid chloroformate, trans-phenylcyclohexanol chloroformate, and ferruginol chloroformate.

5. Method according to any one of the preceding claims, characterized in that the product of formula (I) is obtained from a product of formula (II) 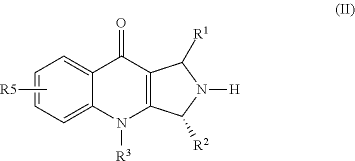
wherein:
R¹ is selected from the group consisting of H, ═O, carboxyl, cyano, amino, and halogen; lower alkyl or alkene or alkenyl or alkoxy or alkylamine comprising 1-6 carbon atoms, unsubstituted or substituted preferably with halogen, cyano, amino, and hydroxy, among others; —C(O)—(C₁₋₆)alkyl, —C(O)—(C₁₋₆)alkoxy, —C(O)—NH—(C₁₋₆)alkylamino or —C(O)—NH₂;
R² is selected from the group consisting of H, ═O, carboxyl, cyano, amino, halogen, trifluoromethyl, trifluoromethoxy, (C₁₋₁₀)alkyl or alkene or alkenyl or alkoxy or alkyl, unsubstituted or substituted preferably with halogen, cyano, amino, hydroxy, trifluoromethyl, and trifluoromethoxy, among others; bipyridine and pyridine substituted with alkyl, alkene, halide, and carboxy; benzyl substituted with alkyl, alkene, halide, carboxy, carbamate, nitro, alkyl, alkene, and carboxy; cycloalkyl or heteroaryl or heterocycloalkyl unsubstituted or substituted with O, S, P, halogen, cyano, amino, hydroxy, lower aryl, alkyl or alkene or alkenyl or alkoxy or lower alkylamine, trifluoromethyl, and trifluoromethoxy, among others;
R³ is selected from CH₃; H; lower alkyl or alkene or alkynyl or carbonyl alkyl, carbonyl alkynyl or carbonyl alkene; sulfates; phenyl substituted with alkyl, alkene, halide, and carboxy; pyridine and bipyridine substituted with alkyl, alkene, halide, and carboxy; benzyl substituted with alkyl, alkene, halide, carboxy, carbamate, nitro, alkyl, alkene, and carboxy among others;
Y may be selected from a bond, lower alkyl or alkenyl or alkynyl, or a heteroatom, phosphates or phosphites;
R⁴ is selected from the group consisting of pyridine and bipyridine substituted with alkyl, alkene, halide, carboxy, chlorobenzyl substituted with alkyl, alkene, halide, carboxy, carbamate, and nitro; chloroalkyl; benzyl substituted with alkyl, alkene, halide, carboxyl, carbamate and nitro; alkyl; alkene; carboxy, chloroalkyl, and chloroalkene; acid chlorides; H, halogen, hydroxy, carboxy, oxo, nitro, lower alkyl or alkene or alkynyl or carbonyl alkyl or carbonyl alkene or carbonyl alkenyl or arylalkyl, heteroaryl, and heterocycloalkyl, whether substituted or not; Boc, Bn, phenyl or benzyl, whether substituted or not, phenylsulfonyl, and naphthyl;
R⁵ may be from 1 to 4 identical or different radicals selected from H, ═O, carboxy, cyano, amino, halogen, trifluoromethyl, trifluoromethoxy, (C₁₋₁₀) alkyl or alkene or alkenyl or alkoxy or alkylamine, unsubstituted or substituted preferably with halogen, cyano, amino, hydroxy, trifluoromethyl, and trifluoromethoxy;
wherein, in addition, R¹, R², R³, R⁴, and R⁵ are selected from:

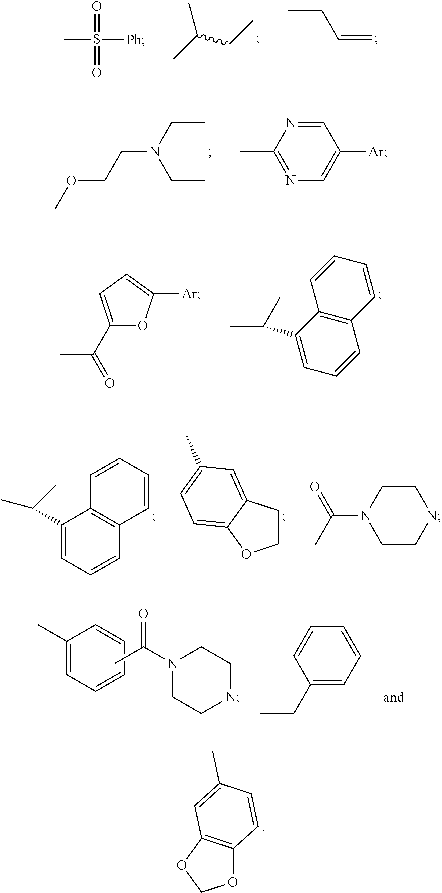

6. Method according to any one of the preceding claims, characterized in that the product of formula (I) is obtained from a product of formula (II) by introduction of (R⁴) radicals using microwaves, wherein the precursors of said (YR⁴) radicals are chlorides.

7. Method according to any one of the preceding claims, characterized in that the Y—R⁴ precursor is chloropyridine and chlorobipyridine substituted with alkyl, alkene, halide, carboxy, chlorobenzyl substituted with alkyl, alkene, halide, carboxy, carbamate, and nitro; chloroalkyl; benzyl substituted with alkyl, alkene, halide, carboxy, carbamate, and nitro; alkyl; alkene; carboxy; chloroalkyl; chloroalkene; acid chloride; H, halogen, hydroxy, carboxy, oxo, nitro, substituted or unsubstituted lower alkyl or alkene or alkynyl or carbonyl alkenyl or carbonyl alkene or carbonyl alkynyl or arylalkyl, heteroaryl, and heterocycloalkyl; Boc, Bn, phenyl or benzyl, whether substituted or not, phenylsulfonyl, and naphthyl.

8. Method according to any one of the preceding claims, characterized in that the product of formula (I) is obtained from a product of formula (II), wherein, in addition, a PdCl₂ catalyzed Buchwald-Hartwig reaction is used along with ionic liquids such as [bmim]Z (wherein Z is selected from PF₆, CF₃CO₂, BF₄, InCl₄, and BPh₄), according to the following scheme:

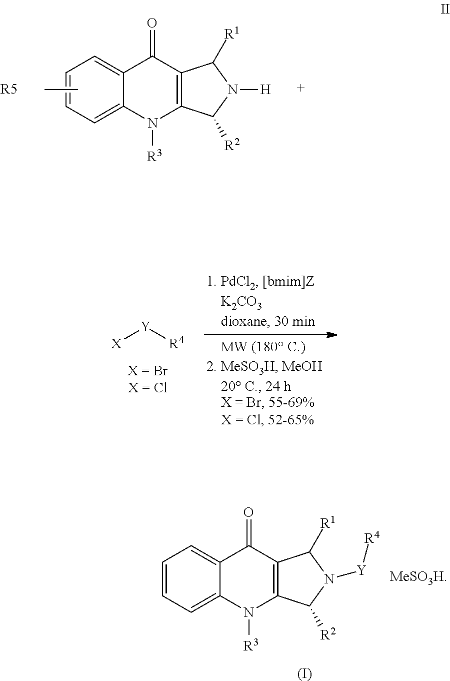

9. Method according to any one of the preceding claims, characterized in that the yield is above 60%, preferably above 75%, more preferably above 90%, and preferably above 99% of the preferred enantiomer, thereby increasing the total amount of the final product by 200% as compared with other methods generally performed.

10. Intermediate compounds of formula (II) 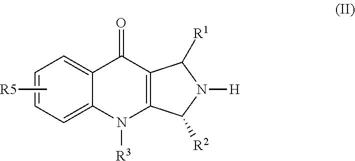
characterized in that the radicals are selected from:
R¹ is selected from the group consisting of H, ═O, carboxyl, cyano, amino, and halogen; lower alkyl or alkene or alkenyl or alkoxy or alkylamine comprising 1-6 carbon atoms, unsubstituted or substituted preferably with halogen, cyano, amino, and hydroxy, among others; —C(O)—(C₁₋₆)alkyl, —C(O)—(C₁₋₆)alkoxy, —C(O)—NH—(C₁₋₆)alkylamino or —C(O)—NH₂;
R² is selected from the group consisting of H, ═O, carboxyl, cyano, amino, halogen, trifluoromethyl, trifluoromethoxy, (C₁₋₁₀)alkyl or alkene or alkenyl or alkoxy or alkyl, unsubstituted or substituted preferably with halogen, cyano, amino, hydroxy, trifluoromethyl, and trifluoromethoxy, among others; bipyridine and pyridine substituted with alkyl, alkene, halide, and carboxy; benzyl substituted with alkyl, alkene, halide, carboxy, carbamate, nitro, alkyl, alkene, and carboxy; cycloalkyl or heteroaryl or heterocycloalkyl unsubstituted or substituted with O, S, P, halogen, cyano, amino, hydroxy, lower aryl, alkyl or alkene or alkenyl or alkoxy or lower alkylamine, trifluoromethyl, and trifluoromethoxy, among others;
R³ is selected from CH₃; H; lower alkyl or alkene or alkynyl or carbonyl alkyl, carbonyl alkynyl or carbonyl alkene; sulfates; phenyl substituted with alkyl, alkene, halide, and carboxy; pyridine and bipyridine substituted with alkyl, alkene, halide, and carboxy; benzyl substituted with alkyl, alkene, halide, carboxy, carbamate, nitro, alkyl, alkene, and carboxy among others;
Y may be selected from a bond, lower alkyl or alkenyl or alkynyl, or a heteroatom, phosphates or phosphites;
R⁴ is selected from the group consisting of pyridine and bipyridine substituted with alkyl, alkene, halide, carboxy, chlorobenzyl substituted with alkyl, alkene, halide, carboxy, carbamate, and nitro; chloroalkyl; benzyl substituted with alkyl, alkene, halide, carboxyl, carbamate and nitro; alkyl; alkene; carboxy, chloroalkyl, and chloroalkene; acid chlorides; H, halogen, hydroxy, carboxy, oxo, nitro, lower alkyl or alkene or alkynyl or carbonyl alkyl or carbonyl alkene or carbonyl alkenyl or arylalkyl, heteroaryl, and heterocycloalkyl, whether substituted or not; Boc, Bn, phenyl or benzyl, whether substituted or not, phenylsulfonyl, and naphthyl;
R⁵ may be from 1 to 4 identical or different radicals selected from H, ═O, carboxy, cyano, amino, halogen, trifluoromethyl, trifluoromethoxy, (C₁₋₁₀) alkyl or alkene or alkenyl or alkoxy or alkylamine, unsubstituted or substituted preferably with halogen, cyano, amino, hydroxy, trifluoromethyl, and trifluoromethoxy;
wherein, in addition, R¹, R², R³, R⁴, and R⁵ are selected from:

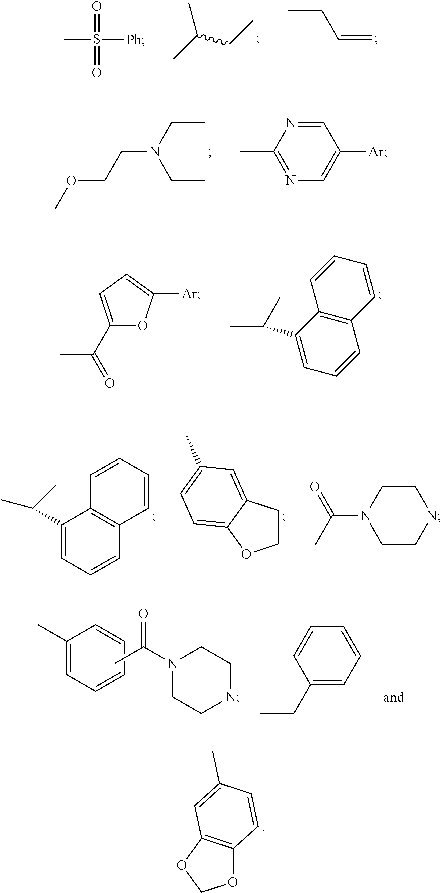

11. Method of obtaining an intermediate compound of formula (II) according to claim 10, characterized in that it is an enantioselective and diastereoselective process for obtaining an enantiomerically enriched product, wherein a hydrogenation reaction is carried out with chiral auxiliaries, thereby obtaining the desired enantiomeric product without requiring resolution of the racemic mixture of β-carbolines by chiral separation of salts and complexes.

12. Method according to claim 11, characterized in that palladium or ruthenium hydride and/or nickel boride are used in the synthesis to reduce the chiral intermediates.

13. Method according to any one of the preceding claims, characterized in that chloroformate chiral auxiliaries are used for the reduction and asymmetric hydrogenation of imines.

14. Method according to any one of the preceding claims, characterized in that the chloroformate chiral auxiliaries are selected from 8-phenyl-menthol, mentholyl chloroformate, abietane chloroformate, ciperenoic acid chloroformate, trans-phenylcyclohexanol chloroformate, and ferruginol chloroformate.

15. Method according to any one of the preceding claims, characterized in that, in a preferred embodiment, the method comprises the following steps: 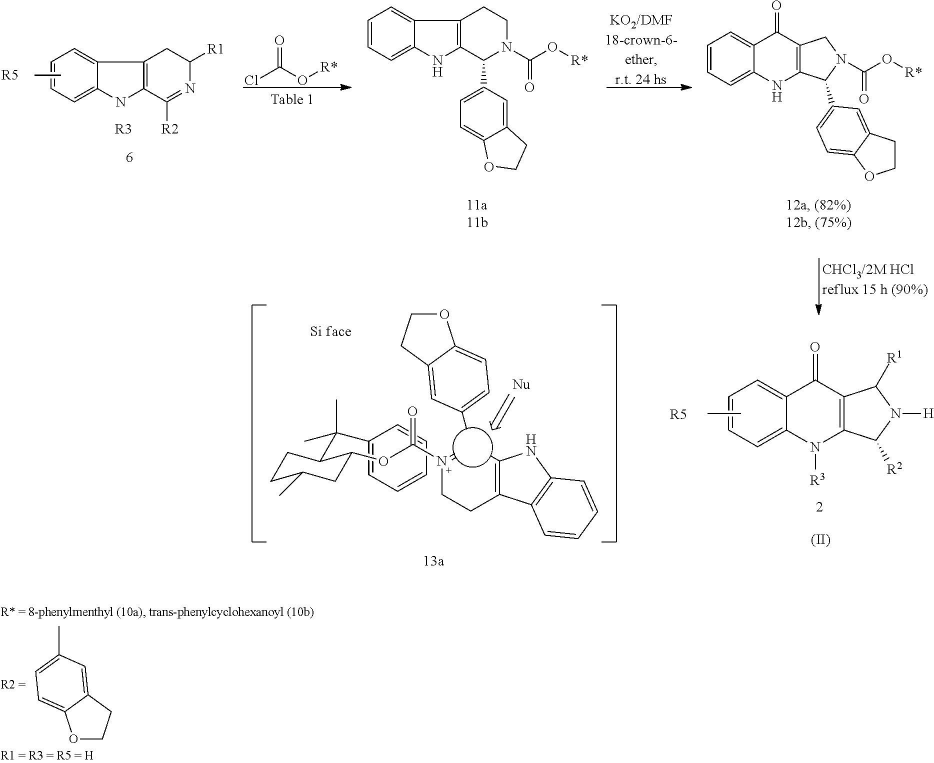
wherein R¹, R², R³ and R⁵ are as described above for the intermediate product of formula (II).
